(19) United States
(12) Patent Application Publication
(10) Pub. No.: US 2008/0319507 A1
(43) Pub. Date: Dec. 25, 2008

(54) Tissue Treatment Device and Method of Restricting Use of Device

(75) Inventor: Thomas R. Myers, Palo Alto, CA (US)

Correspondence Address: RELIANT / FENWICK;c/o FENWICK & WEST, LLP, 801 CALIFORNIA STREET, MOUNTAIN VIEW, CA, 94041, US

(73) Assignee: RELIANT TECHNOLOGIES, INC., Mountain View, CA (US)

(21) Appl. No.: 12/144,627

(22) Filed: Jun. 24, 2008

Related U.S. Application Data
(60) Provisional application No. 60/946,121, filed on Jun. 25, 2007.

(51) Int. Cl. A61N 1/00; A61N 1/08

(52) U.S. Cl. 607/50; 607/59

(57) ABSTRACT

Embodiments include electromagnetic energy-based tissue treatment devices which comprise biometric sensors in combination with memory storing biometric data from authorized treatment providers, and methods of restricting use of the devices based on comparing biometric data from potential treatment providers to the stored biometric data from authorized treatment providers. Delivery of a treatment can be permitted when the biometric data from a potential treatment provider is found to match the biometric treatment data for an authorized treatment provider. The devices of the invention can further be configured to collect usage data and usage setting of the device, and usage data can be used in conjunction with biometric data to determine whether or not an authorized user has exceeded or will exceed a recommended treatment regimen.

### CROSS-REFERENCE TO RELATED APPLICATION

This application claims priority under 35 U.S.C. §119(e) to U.S. Provisional Patent Application Ser. No. 60/946,121, “Tissue treatment device and method of restricting use of device,” filed Jun. 25, 2007. The subject matter of all of the foregoing is incorporated herein by reference in its entirety.

### FIELD OF THE INVENTION

This invention relates generally to electromagnetic energy based tissue treatment devices and methods of using the devices. More particularly, it relates to electromagnetic energy based tissue treatment devices which comprise biometric sensors in combination with memory storing biometric data from authorized treatment providers, and methods of restricting use of the devices based on biometric data.

### BACKGROUND OF THE INVENTION

Forms of electromagnetic energy, such as electrical energy, thermal energy, optical energy and radiofrequency energy, are commonly used as versatile tools in medicine to achieve desired outcomes in a tissue that is treated. For example, forms of electromagnetic energy have been used to treat common dermatological problems such as hypervascular lesions, pigmented lesions, acne scars, rosacea, for hair removal, etc. Additionally, forms of electromagnetic energy are also used for cosmetic purposes for achieving a better cosmetic appearance by resurfacing the skin and remodeling the different layers of skin to improve the appearance of wrinkled or aged skin and/or to tighten skin. For example, in laser skin remodeling, laser energy penetrates into at least a portion of the deeper layers of the skin and is aimed at stimulating the generation of and/or altering the structure of extra-cellular matrix materials, such as collagen, that contribute to the youthful appearance of skin.

As cosmetic treatments using these devices are becoming increasingly popular, the desire to be able to use these devices in the home is increasing as well. For example, the HairMax™ LaserComb, Lexington International LLC, Boca Raton, Fla. USA, which uses low level laser therapy (LLLT) to improve the appearance of hair, is marketed directly to consumers. Another example of an electromagnetic energy based tissue treatment device is the Zeno® acne clearing device, Tyrell Inc., Houston, Tex. USA, which uses heat to treat acne in the home. The NūFace® Micro-Current Delivery System, Skin Star, Inc., Encinitas, Calif. USA is a home device used to deliver microcurrent impulses to strategic locations under the surface of the skin to diminish minor lines and wrinkles, brighten skin, reduce puffiness, and tighten and tone facial muscles.

The majority of devices currently marketed for home use low treatment energies and thus may not pose significant safety risks when used by unintended or unauthorized users (e.g., children) or when used more frequently than suggested. However, increased demand for effective treatments that can be provided in a spa or home setting may lead to higher energy devices being marketed directly to individuals lacking medical training, such as spa treatment providers or consumers. Such higher energy devices will require systems which can ensure the devices are activated only by authorized treatment providers and which can ensure the devices are used in a safe manner.

### SUMMARY OF THE INVENTION

The devices of the present invention restrict use of the devices to authorized treatment providers who are identified using biometric data. The devices of the present invention can further restrict use by an authorized treatment provider based on recommended treatment frequencies and/or durations. The present invention is directed to devices configured to deliver electromagnetic energy which use biometric sensors and memory storing biometric data from authorized treatment providers in order to restrict use of the devices only to authorized treatment providers, and to prohibit use of the device by unauthorized treatment providers. In one example, the authorized treatment provider can also be the individual receiving the treatment. The devices of the present invention can further record information about the frequency and/or duration of use of the device by an authorized treatment provider, and can prevent usage of the device by an authorized treatment provider that exceeds a recommended treatment regimen. The present invention is also directed to methods of restricting use of these devices to authorized treatment providers by collecting biometric data from a potential treatment provider, comparing the biometric data to stored biometric data of authorized treatment providers, determining whether or not the biometric data matched that of an authorized treatment provider within a pre-determined tolerance, and enabling use of the device only when the biometric data is found to match within a pre-determined tolerance. The methods of the present invention can further comprise the steps of checking the frequency and/or duration of one or more previous usages of the device by an authorized treatment provider, determining whether or not the one or more previous usages have exceeded a recommended usage level, determining whether or not the one or more previous usages in combination with the current potential usage will exceed a recommended usage level, and enabling use of the device only when both the biometric data is found to match within a pre-determined tolerance, and the previous usage and/or previous usage and potential current usage levels are within or below a recommended usage level.

In one embodiment, the present invention is directed to a tissue treatment device, comprising: at least one electromagnetic energy source configured to produce electromagnetic energy, at least one controller configured to control the at least one electromagnetic energy source, at least one handpiece configured to direct the electromagnetic energy to a tissue in order to provide a treatment, at least one biometric sensor configured to detect at least one piece of biometric data from a potential treatment provider, and at least one memory storing at least one piece of biometric data from at least one authorized treatment provider, wherein the device is configured to enable treatment only when the at least one piece of biometric data from the potential treatment provider is authenticated by comparing it to the least one piece of biometric data from the at least one authorized treatment provider, thereby preventing use of the device by one or more unauthorized treatment providers.

In another embodiment, the present invention is directed to a method of restricting the use of a tissue treatment device to at least one authorized treatment provider, comprising: providing a tissue treatment device, wherein the tissue treatment device comprises at least one electromagnetic energy source configured to produce electromagnetic energy, at least one controller configured to control the at least one electromagnetic energy source, at least one handpiece configured to direct the electromagnetic energy to a tissue in order to provide a treatment, at least one biometric sensor configured to detect at least one set of biometric data from an potential treatment provider; and at least one memory configured to store at least one set of biometric data from at least one authorized treatment provider; detecting at least one piece of biometric data from the potential treatment provider; comparing the at least one set of biometric data from the potential treatment provider to at least one set of biometric data from at least one authorized treatment provider; determining whether or not the at least one set of biometric data from the potential treatment provider matches any of the at least one set of biometric data of the at least one authorized treatment provider within a pre-determined tolerance; and enabling delivery of the treatment when the at least one set of biometric data from the potential treatment provider matches any of the at least one set of biometric data of the at least one authorized treatment provider within the pre-determined tolerance.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a drawing illustrating a device configured to deliver electromagnetic energy which comprises a biometric sensor and memory.

FIGS. 2A and 2B illustrate embodiments of a device configured to deliver electromagnetic energy which comprises a biometric sensor, memory, and a switch for turning on and off the delivery of electromagnetic energy. In FIG. 2A, the switch is controlled by a foot pedal. In FIG. 2B the switch is located on the handpiece.

### DETAILED DESCRIPTION

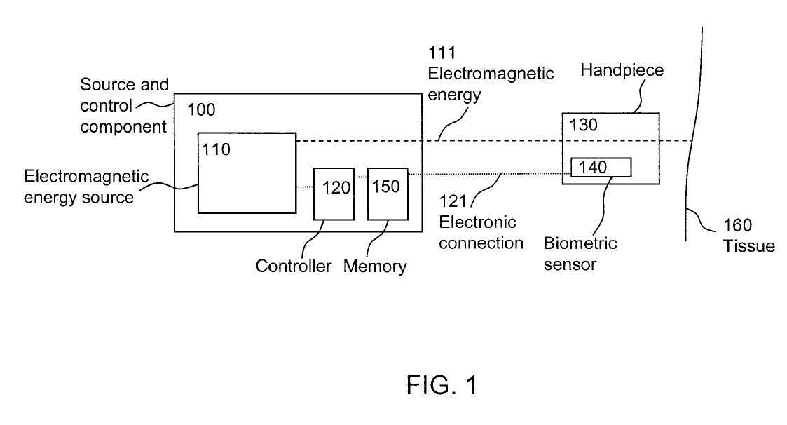

FIG. 1 is a drawing illustrating a device configured to deliver electromagnetic energy which comprises an electromagnetic energy source 110, a controller 120, a handpiece 130, a biometric sensor 140, and memory for the biometric sensor 150. The electromagnetic energy 111 is produced in the source and control component 100 of the device by the electromagnetic energy source 110, and is directed from the source and control component 100 to the handpiece 130 which then serves to deliver the electromagnetic energy 111 to a tissue 160 which is to be treated. Electrical connections 121 connect the biometric sensor 140, the controller 120, the electromagnetic energy source 110, and the memory 150, which can be housed in the source and control component 100 as shown, or in the handpiece 130. When the components of the device are operably coupled, the controller 120 can be used to control the electromagnetic energy source 110, can be used to collect and process data from the biometric sensor 140, can be used to access data stored in the memory 150, can be used to compare data collected from the biometric sensor 140 with data stored in the memory 150, and can be used to enable use of the device based on the results of the data comparison.

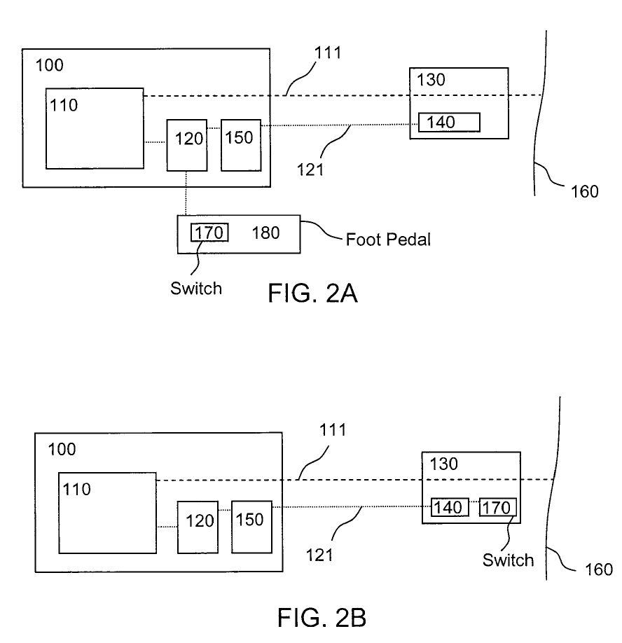

FIGS. 2A and 2B, illustrate two examples of devices comprising switches 170 which can be used to control delivery of the electromagnetic energy 111. In FIGS. 1, 2A and 2B, like elements are labeled using the same reference numerals.

FIG. 2A illustrates a device configured to deliver electromagnetic energy 111 which comprises an electromagnetic energy source 110, a controller 120, a handpiece 130, a biometric sensor 140, memory for the biometric sensor 150, and a foot pedal 180 containing a switch 170 which can be used to turn on and off the delivery of the electromagnetic energy 111 through the handpiece 130 which is activated by the use of the foot pedal. In this example, when the components of the device are operably coupled, the controller 120 can be used to control the electromagnetic energy source 110, can be used to collect and process data from the biometric sensor 140, can be used to access data stored in the memory 150, can be used to compare data collected from the biometric sensor 140 with data stored in the memory 150, and, when the results of the data comparison indicate that an authorized treatment provider is the potential treatment provider, can be used to enable the switch 170in the foot pedal 180 to turn on the delivery of the electromagnetic energy 111 through the handpiece 130 in order to treat a portion of tissue 160.

The drawing FIG. 2B illustrates a device configured to deliver electromagnetic energy 111 which comprises an electromagnetic energy source 110, a controller 120, a handpiece 130, a biometric sensor 140, memory for the biometric sensor 150, and a switch 170 located in the handpiece 130 which can be used to turn on and off the delivery of the electromagnetic energy 111 through the handpiece 130. In this example, when the components of the device are operably coupled, the controller 120 can be used to control the electromagnetic energy source 110, can be used to collect and process data from the biometric sensor 140, can be used to access data stored in the memory 150, can be used to compare data collected from the biometric sensor 140 with data stored in the memory 150, and, when the results of the data comparison indicate that an authorized treatment provider is the potential treatment provider, can be used to enable the switch 170 in the handpiece 130 to turn on the delivery of the electromagnetic energy 111 through the handpiece 130 in order to treat a portion of tissue 160.

In one example of the device, the at least one electromagnetic energy source 110 can be selected from the group consisting of an optical energy source, a radiofrequency source, a thermal energy source, an electrical energy source, and combinations thereof. In another example, the at least one electromagnetic energy source 110 can be a laser energy source. In yet another example, the device can be configured to deliver optical energy in a fractional manner.

Devices which deliver electromagnetic energy in order to treat a tissue, such as, for example, devices which deliver medical and/or cosmetic tissue treatments, typically include a delivery apparatus of some type. One example of a delivery apparatus is a handpiece. While generally a handpiece is designed to be held in the hand of a user or a treatment provider (e.g., a physician, a medical professional, a cosmetic professional, a consumer, etc.) and used to apply energy to a tissue, for the purposes of the present invention, a handpiece will be understood more broadly to mean any apparatus which is configured to be used to deliver electromagnetic energy to a tissue. In one example, a handpiece 130 can be configured to be used by a treatment provider to direct electromagnetic energy 111 from an electromagnetic energy source 110 to a tissue 160 to be treated so as to deliver a safe and effective treatment to the tissue 160.

The biometric sensor 140 can be located in the handpiece 130 or in another component of the devices of the present invention. Optionally, when the biometric sensor 140 is located in the handpiece 130, the handpiece 130 can further comprise an optical energy delivery system which directs the optical energy. Optionally, a handpiece 130 can further comprise a memory 150 storing at least one piece of biometric data. The memory 150 can further store other types of data, such as, for example, usage data, characteristic data describing the handpiece 130, describing a beam and/or a beam path through the handpiece 130, etc. Optionally, a handpiece 130 can be configured to be repeatedly connected to, disconnected from, and reconnected to one or more source and control components of a device, and/or to one or more electromagnetic energy sources 110. A handpiece 130 can have at least one ingressive end where the electromagnetic energy 111 comes into the handpiece 130 and at least one emissive end where the electromagnetic energy 111 leaves the handpiece 130. During use, the handpiece 130 can be either held steady in order to deliver the electromagnetic energy 111, or can be in motion while the electromagnetic energy 111 is delivered. In one example, a handpiece 130 can be a maneuverable handpiece 130 sized for manipulation by a human hand.

In one example, the controller 120 can be housed in a source and control component 100 as shown in FIGS. 1, 2A and 2B. Alternately, the controller 120 can be housed in a separate component, or in the handpiece 130. The controller 120 can comprise a microprocessor and/or a digital signal processor (DSP), as well as software, firmware, algorithms, a look-up table, etc. The controller 120 can be configured to control functional parameters of the electromagnetic energy source 110. The controller 120 can be configured to be in communication with the biometric sensor 140 and/or with the memory 150 storing at least one piece of biometric data. The controller 120 can be configured to access the biometric data stored in the memory 150. The controller 120 can be configured to authenticate the biometric data collected by the biometric sensor 140 by comparing it with biometric data stored in the memory 150. The controller 120 can be configured to encrypt biometric data and/or to read encrypted biometric data. Optionally, the controller 120 can be configured to be in communication with one or more switches, such as switch 170.

As previously discussed, the devices of the present invention comprise one or more biometric sensors 140. A biometric characteristic or trait is a measurable physical characteristic or personal behavioral trait that can be used to recognize and/or authenticate the claimed identity of an individual. A biometric sensor is a sensor that is configured to detect and/or capture a sample of a biometric characteristic or trait from an individual. Biometric data can be extracted from a biometric sample in order to construct a reference template. The biometric data and/or the reference template can then be used in various matching scenarios in order to try to authenticate the identity of an individual.

There are numerous types of biometric technologies that are commonly used and have been incorporated into automated biometric technologies. For example, body odor recognition, DNA recognition, earlobe recognition, finger imaging, finger geometry recognition, facial recognition, facial thermogram recognition, hand geometry recognition, iris recognition, gait recognition, live grip recognition, palm recognition, retinal recognition, signature verification, skin print recognition, vein recognition, voice authentication, etc.

For example, the biometric sensor 140 can be a capacitance sensor, a temperature sensor, an optical scanner, a touchpad, a slide sensor, or combinations thereof. Alternatively or additionally, the biometric sensor 140 can be a facial recognition system, an iris recognition system, a pupil size recognition system, a voice recognition system, a fingerprint recognition system, a palmprint recognition system, a handprint recognition system, or combinations thereof. In another example, the biometric sensor 140 can be a charge coupled device (CCD) that records images electronically. In another example, the biometric sensor 140 can be a complementary metal oxide semiconductor (CMOS). In another example, the biometric sensor 140 can be a capacitance-based fingerprint sensor. In yet another example, the biometric sensor 140 can be a capacitance-based fingerprint sensor in combination with a temperature sensor.

In one example, the sensing surface of the biometric sensor 140 can be located in the handpiece 130. In another example, the biometric sensor 140 can be configured to detect at least one piece of biometric data when the handpiece 130 is grasped by the potential treatment provider. In yet another example, the biometric sensor 140 can be configured to detect the continued presence of an authorized treatment provider in order to continue a treatment.

In one example, the biometric sensor 140 can be configured to detect one piece of biometric data. In another example, the biometric sensor 140 can be configured to detect a set of biometric data. In another example, the at least one piece of biometric data from the potential treatment provider and/or the at least one piece of biometric data from the at least one authorized treatment provider can be a fingerprint, a palmprint, a handprint, a voice record, a record of iris color, a record of iris pattern, a record of iris structure, a record of pupil size, a record of facial structure, and combinations thereof.

The memory 150 storing biometric data can be, for example, an EPROM or an EEPROM. The memory 150 can be a separate and stand-alone memory element, or can be part of another component, such as, for example, the controller 120, a security chip, a control chip, a processor, a microprocessor, etc. In one example, the memory 150 can be encrypted memory. In another example, the stored at least one piece of biometric data can be encrypted biometric data. In yet another example, the communication between the controller 120 and the memory 150 can be encrypted.

In one example, the controller 120 and/or the memory 150 can be configured to record the frequency and/or duration of the use of the device by the authorized treatment provider. For example, a time of use, a date of use, and/or a duration of use of the device can be recorded. The recorded frequency and/or duration of use of the device by the authorized treatment provider can be evaluated to determine whether or not a potential current usage and/or previous usage by the authorized treatment provider have exceeded a recommended treatment regime. A recommended treatment regime can be described, for example, as the recommended number of treatments per day, the recommended number of treatments per week, the recommended duration of treatment in a day, the recommended duration of treatment in a week, the recommended total duration of treatment, etc.

In another example, the controller 120 and/or memory 150 can be configured to associate a set of treatment parameters or a range of possible treatment parameters, or a level of possible treatment parameters with an authorized treatment provider or with a group of authorized treatment providers. For example, the controller 120 and/or memory 150 can be configured such that different treatment levels are established (e.g., a level where only low power treatments are enabled by the device, a level where only low and medium power treatments are enabled, a level where low, medium and high power treatments are enabled, a training level where the device does not fire the source of electromagnetic energy, etc.) Individual treatment providers or groups of treatment providers can then have one or more possible treatment levels assigned to them. For example, in-home treatment providers can be assigned to the low power level, aestheticians and spa workers can be assigned to the medium power level, physicians and nurses can be assigned the high treatment level, treatment providers in training can be assigned to the training level, etc. At the time the device authenticates the authorized user, it can then automatically set the range or level of possible treatment parameters based on the level of the authorized user.

In another embodiment, the device can further comprise a system for a potential treatment provider to use to enter a password. In this example, the at least one memory 150 of the device can contain at least one password for the at least one authorized treatment provider, and the device can be configured to enable treatment only when both the at least one piece of biometric data from the potential treatment provider is authenticated by comparing it to the least one piece of biometric data from the at least one authorized treatment provider, and the password entered by the potential treatment provider is authenticated by comparing it to the at least one password for the at least one authorized treatment provider, thereby preventing use of the device by one or more unauthorized treatment providers. In a further example, the memory 150 can contain an encryption algorithm, and the at least one password for the at least one authorized treatment provider stored in memory 150 can be encrypted.

In one example, the device can further comprise a system for entering data on individual patients treated using the device. The device can be configured to record the treatment parameters and/or the treatment settings used to treat one or more individual patients, such as, for example, the treatment level, the power, the fluence, the treatment density, etc. which were used to deliver a treatment to an individual. In another example, the device can further be configured to record the date of a treatment of an individual patient, for example, to track the total treatment parameters used over a series of treatments given to an individual patient over time, and to evaluate whether or not an individual patient has exceeded a recommended treatment regimen. In yet another example, when the authorized treatment provider is also the individual receiving the treatment, the treatment settings used to treat the authorized treatment provider can be correlated with the biometric data for that authorized treatment provider, and the controller 120 can be configured to automatically set the treatment settings to be the treatment settings previously used by an authorized treatment provider when the device determines that the potential treatment provider is the authorized treatment provider.

As illustrated in FIG. 2A and FIG. 2B, the device of the present invention can further comprise a switch 170. The switch 170 can be used by the authorized treatment provider to turn on and off delivery of the electromagnetic energy 111 when the at least one set of biometric data from the potential treatment provider has been found to match the at least one set of biometric data from the at least one authorized treatment provider. In one example, the switch can be a switch 170 located in the handpiece 130 or in a foot pedal 180.

The device of the present invention can further comprise a manual override system which allows the biometric sensor 140 to be bypassed in order to enable delivery of a treatment. The device of the present invention can further comprise one or more power sources. In one example, the power source can be rechargeable, such as, for example, a rechargeable battery.

The device of the present invention can be, for example, a dermatological treatment device for use on human skin. The treatment provider can be, for example, a physician, a medical professional, a cosmetic professional, an aesthetician, a consumer, etc. In one example, the individual receiving the treatment can be a patient. In one example, the individual receiving the treatment can be a different individual than the treatment provider. In another example, the treatment provider and the individual receiving the treatment can be the same individual.

In another embodiment, the method is directed to a method of restricting the use of a device to at least one authorized treatment provider. The method can comprise the steps of: providing a tissue treatment device, wherein the tissue treatment device comprises at least one electromagnetic energy source 110 configured to produce electromagnetic energy 111, at least one controller 120 configured to control the at least one electromagnetic energy source 110, at least one handpiece 130 configured to direct the electromagnetic energy 111 to a tissue 160 in order to provide a treatment, at least one biometric sensor 140 configured to detect at least one set of biometric data from a potential treatment provider; and at least one memory 150 storing at least one set of biometric data from at least one authorized treatment provider; detecting at least one set of biometric data from the potential treatment provider; comparing the at least one set of biometric data from the potential treatment provider to at least one set of biometric data from at least one authorized treatment provider; determining whether or not the at least one set of biometric data from the potential treatment provider matches any of the at least one set of biometric data of the at least one authorized treatment provider within a pre-determined tolerance; and enabling delivery of the treatment when the at least one set of biometric data from the potential treatment provider matches any of the at least one set of biometric data of the at least one authorized treatment provider within the pre-determined tolerance. In one example, the method of the present invention can further comprise the step of delivering the treatment to an individual who may or may not also be the treatment provider.

The process of using a biometric sensor 140 can, for example, comprise the steps of: detecting biometric data, capturing, digitizing and entering the biometric data into a database, extracting the biometric data in order to create a reference template that is unique to an individual, comparing a new sample of biometric data to biometric data and/or a reference template already entered into memory 150, and determining whether or not the new sample of biometric data matches biometric data and/or a reference template already entered into memory 150. The sample of biometric data can be a set of biometric data, or can be one or more pieces of biometric data.

In one example of the method, the step of comparing can be comparing one piece and/or set of biometric data from the potential treatment provider to one piece and/or set of biometric data from the at least one authorized treatment provider, comparing one piece and/or set of biometric data from the potential treatment provider to multiple pieces and/or sets of biometric data from the at least one authorized treatment provider, comparing multiple pieces and/or sets of biometric data from the potential treatment provider to one piece and/or set of biometric data from the at least one authorized treatment provider, or comparing multiple pieces and/or sets of biometric data from the potential treatment provider to multiple pieces and/or sets of biometric data from the at least one authorized treatment provider.

In one example of the method, the at least one piece of biometric data must be detected repeatedly or essentially continuously in order to allow the delivering of the treatment to continue. In another example, the method can further comprise the step of the authorized treatment provider maintaining contact with the at least one biometric sensor 140 of the provided device in order to continue deliver of the treatment.

The method can further comprise the step of recording data describing the frequency and/or duration of a treatment in memory 150 or in another memory communicatively coupled to the device. For example, the step can be the step of recording an amount of time the device is in use, recording a time of day the device is in use, recording the date the device is in use, etc. The step can be the step of comparing the actual frequency and/or duration of use with a recommended treatment regimen. For example, the step can further include the step of comparing an amount of time, a time of day, and/or a date the device was previously used by an authorized treatment provider to a recommended amount of treatment time and/or frequency of use, and the determining step can further include determining whether or not an authorized treatment provider has exceeded the recommended amount of treatment time and/or frequency of use. By recording data describing the frequency and/or duration of a treatment, the device can further restrict use of the device to authorized treatment providers whose usage of the device is within or below recommended treatment regimens.

The method can further comprise the step of storing in memory 150 or another memory at least one treatment parameter and/or treatment setting used in treating an individual. The individual may or may not be an authorized treatment provider. The method can further comprise the step of automatically adjusting the treatment settings for the device to match the treatment settings used the last time the authorized treatment provider used the device.

The method step of enabling delivery can be enabling use of a switch to turn on and off the electromagnetic energy 111 delivery. The method of enabling delivery can be enabling a switch 170 to deliver power to a component of the device, such as, for example, to the controller 120, to the electromagnetic energy source 110, etc.

In one example, the processes of collecting biometric data, accessing biometric data, and/or comparing biometric data can involve using an encryption system, an encryption algorithm, and/or using encrypted data.

The method step of determining whether or not the at least one set of biometric data from the potential treatment provider matches any of the at least one set of biometric data of the at least one authorized treatment provider within a pre-determined tolerance can further comprise determining whether or not a password entered by the potential treatment provider matches any of a set of at least one passwords for the at least one authorized treatment provider stored in the memory 150. In one example, the password entered by the potential treatment provider and/or the set of at least one passwords stored in the memory 150 can be encrypted.

Although the detailed description contains many specifics, these should not be construed as limiting the scope of the invention but merely as illustrating different examples and aspects of the invention. It should be appreciated that the scope of the invention includes other embodiments not discussed in detail above. Various modifications, changes and variations which will be apparent to those skilled in the art may be made in the arrangement, operation and details of the methods and devices of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

In the specification and in the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

1. A tissue treatment device, comprising:
at least one electromagnetic energy source configured to produce electromagnetic energy,
at least one controller configured to control the at least one electromagnetic energy source,
at least one handpiece configured to direct the electromagnetic energy to a tissue in order to provide a treatment,
at least one biometric sensor configured to detect at least one piece of biometric data from a potential treatment provider, and
at least one memory storing at least one piece of biometric data from at least one authorized treatment provider,
wherein
the device is configured to enable treatment only when the at least one piece of biometric data from the potential treatment provider is authenticated by comparing it to the least one piece of biometric data from the at least one authorized treatment provider, thereby preventing use of the device by one or more unauthorized treatment providers.

2. A method of restricting the use of a tissue treatment device to at least one authorized treatment provider, comprising:
providing a tissue treatment device, wherein the tissue treatment device comprises
at least one electromagnetic energy source configured to produce electromagnetic energy,
at least one controller configured to control the at least one electromagnetic energy source,
at least one handpiece configured to direct the electromagnetic energy to a tissue in order to provide a treatment,
at least one biometric sensor configured to detect at least one set of biometric data from an potential treatment provider; and
at least one memory storing at least one piece of biometric data from at least one authorized treatment provider;
detecting at least one piece of biometric data from the potential treatment provider;
comparing the at least one piece of biometric data from the potential treatment provider to at least one piece of biometric data from at least one authorized treatment provider;
determining whether the at least one piece of biometric data from the potential treatment provider matches any of the at least one piece of biometric data of the at least one authorized treatment provider within a pre-determined tolerance; and
enabling delivery of the treatment when the at least one piece of biometric data from the potential treatment provider matches any of the at least one piece of biometric data of the at least one authorized treatment provider within the pre-determined tolerance.
